(19) United States
(12) Patent Application Publication
(10) Pub. No.: US 2013/0196092 A1
(43) Pub. Date: Aug. 1, 2013

(54) ANTIPERSPIRANT COMPOSITIONS HAVING PARTICULATE OPACIFYING AGENT, ANTIPERSPIRANT PRODUCTS INCLUDING THE ANTIPERSPIRANT COMPOSITIONS, AND METHODS OF PREPARING THE ANTIPERSPIRANT COMPOSITIONS

(71) Applicant: Berea Thompson, Somerset, NJ (US)

(72) Inventor: Berea Thompson, Somerset, NJ (US)

(73) Assignee: The Dial Corporation, Scottsdale, AZ (US)

(21) Appl. No.: 13/361,189

(22) Filed: Jan. 30, 2012

(51) Int. Cl. A61K 8/28; A61Q 15/00; A61K 8/02; B32B 1/02; A61K 8/26; A61K 8/27

(52) U.S. Cl. 428/34.1; 424/65; 424/68; 424/66; 424/67; 424/401

(57) ABSTRACT

An antiperspirant composition includes an antiperspirant active compound and a particulate opacifying agent. The antiperspirant active compound is based upon an astringent metallic salt. The particulate opacifying agent has a refractive index greater than a comparable antiperspirant composition absent the particulate opacifying agent, provided that a difference in refractive index between the particulate opacifying agent and the comparable antiperspirant composition absent the particulate opacifying agent is less than or equal to about 0.3. An antiperspirant product including a container and the antiperspirant composition disposed in the container is also provided.

### TECHNICAL FIELD

The present invention generally relates to antiperspirant compositions, antiperspirant products including the antiperspirant compositions, and methods of preparing the antiperspirant compositions. More particularly, the invention relates to antiperspirant compositions having a minimized yellow color and minimized white film upon application to skin of consumers, antiperspirant products including the antiperspirant compositions, and methods of preparing the antiperspirant compositions.

### BACKGROUND

Antiperspirant compositions are well known personal care products used to prevent or eliminate perspiration and body odor caused by perspiration. Antiperspirant compositions come in a variety of forms and may be formulated, for example, into aerosols, pumps, sprays, liquids, roll-ons, lotions, creams, sticks, soft solids, and the like.

Stick antiperspirant products include an antiperspirant composition that is disposed within a container. During use of the stick antiperspirant products, the top of the container is removed and the application surface of the antiperspirant composition is contacted with the skin, such as the underarm, by swiping or rubbing the stick across the skin. Sometimes the stick antiperspirant product also includes an undercap, or factory seal, that covers the application surface and is removed prior to first use. The container generally also includes some mechanism for moving the antiperspirant composition upwards through the container to continue to provide an exposed application surface.

Commercial markets for antiperspirant products are highly competitive, with consumers wanting products that perform well in terms of antiperspirant performance, while minimizing appearance of white film or residue upon application of the antiperspirant compositions to skin of a consumer. White film upon application of the antiperspirant compositions to skin of a consumer is often attributed to a large difference between refractive index of antiperspirant active compounds relative to a refractive index of the antiperspirant compositions as a whole. To reduce white film upon application of the antiperspirant compositions to skin of a consumer, additional components may be added to the antiperspirant compositions to reduce the gap between the refractive indices of the antiperspirant active compounds and the antiperspirant compositions as a whole. One example of a class of such additional components is alkyl benzoates, which generally have a refractive index of greater than 1.4 and enable refractive indices of the antiperspirant active compounds and the antiperspirant compositions to be more closely matched.

Appearance of the antiperspirant compositions prior to application is also a factor in consumer preferences. Clear gel antiperspirant compositions have been developed that avoid off-color appearances. However, many antiperspirant compositions, especially those in solid stick form, cannot be developed with a clear appearance. Instead, white appearance is sought for such antiperspirant compositions, with care taken to avoid yellow color. While alkyl benzoates reduce appearance of white film upon application of the antiperspirant compositions, the alkyl benzoates do not affect appearance of the antiperspirant compositions prior to application. To achieve the white appearance and minimize yellow color, relatively expensive or non-zirconium containing antiperspirant active compounds are generally employed that do not impart yellow color to the antiperspirant compositions. For example, tri- and tetra-chlorohydrate salts of aluminum and zirconium may enable whiteness to be achieved without yellow color in the antiperspirant compositions. When formulating antiperspirant compositions with various common (and cheaper) antiperspirant active compounds, such as compounds having a ratio of aluminum to zirconium of greater than 6.0:1.0 including aluminum zirconium pentachlorohydrate and aluminum zirconium pentachlorohydrex glycine complex, yellow color of the antiperspirant compositions often cannot be avoided.

It is known to include titanium dioxide in antiperspirant compositions as an opacifying agent to enhance whiteness of the antiperspirant compositions. However, titanium dioxide is often attributable to undesirable appearance of white film upon application of the antiperspirant compositions to the skin of consumers.

Accordingly, it is desirable to provide antiperspirant compositions that exhibit an enhanced white appearance with minimal yellow color, even when salts of aluminum and zirconium having a ratio of aluminum to zirconium of greater than 6.0:1.0 are employed as antiperspirant active compounds. It is also desirable to provide antiperspirant compositions that generally exhibit an enhanced white appearance with minimal yellow color, regardless of the type of antiperspirant active compound employed. It is also desirable to provide antiperspirant compositions having a minimized appearance of white film upon application to skin of a consumer. It is also desirable to develop various forms of antiperspirant compositions, including stick and gel forms, which benefit from the enhanced white appearance with minimal yellow color while minimizing appearance of white film upon application to skin of a consumer. Furthermore, other desirable features and characteristics of the present invention will become apparent from the subsequent detailed description of the invention and the appended claims, taken in conjunction with the accompanying drawings and this background of the invention.

### BRIEF SUMMARY

Antiperspirant compositions, antiperspirant products including antiperspirant compositions, and methods of preparing the antiperspirant compositions are provided herein. In an embodiment, an antiperspirant composition includes an antiperspirant active compound and a particulate opacifying agent. The antiperspirant active compound is based upon an astringent metallic salt. The particulate opacifying agent has a refractive index greater than that of a comparable antiperspirant composition absent the particulate opacifying agent, provided that a difference in refractive index between the particulate opacifying agent and the comparable antiperspirant composition absent the particulate opacifying agent is less than or equal to about 0.3.

In another embodiment, an antiperspirant product is provided that includes a container and an antiperspirant composition disposed in the container. The antiperspirant composition includes an antiperspirant active compound and a particulate opacifying agent. The antiperspirant active compound is based upon an astringent metallic salt. The particulate opacifying agent has a refractive index greater than a comparable antiperspirant composition absent the particulate opacifying agent, provided that a difference in refractive index between the particulate opacifying agent and the comparable antiperspirant composition absent the particulate opacifying agent is less than or equal to about 0.3.

In another embodiment, a method of preparing an antiperspirant includes the step of mixing an antiperspirant active compound and a particulate opacifying agent. The antiperspirant active compound is based upon an astringent metallic salt. The particulate opacifying agent has a refractive index greater than a comparable antiperspirant composition absent the particulate opacifying agent, provided that a difference in refractive index between the particulate opacifying agent and the comparable antiperspirant composition absent the particulate opacifying agent is less than or equal to about 0.3.

### BRIEF DESCRIPTION OF THE DRAWINGS

Embodiments of the present invention will hereinafter be described in conjunction with the following drawing figures, wherein like numerals denote like elements, and wherein:

FIG. 1 is a perspective view of an antiperspirant product in accordance with an exemplary embodiment;

FIG. 2 is a top view of the antiperspirant product depicted in FIG. 1; and

FIG. 3 is an exploded perspective view of an antiperspirant product in accordance with an exemplary embodiment.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Antiperspirant compositions, antiperspirant products that include the antiperspirant compositions, and methods of preparing antiperspirant compositions that exhibit minimized yellow color and no or minimal appearance of white film upon application to skin of a consumer are provided. More specifically, the antiperspirant compositions contemplated herein include an antiperspirant active compound and a particulate opacifying agent, and may further include various additional components as known in the art for solid or gel antiperspirant compositions, such as a carrier system and a solidification system for the antiperspirant active compounds and the particulate opacifying agent. The particulate opacifying agent has a refractive index that is greater than the antiperspirant composition in the absence of the particulate opacifying agent, which serves to raise a refractive index of the antiperspirant composition as a whole and, thus, reduces a difference between refractive index of antiperspirant active compound and the antiperspirant composition as a whole. Further, due to the particulate form and refractive index of the opacifying agent, the particulate opacifying agent scatters light to minimized yellow color of the antiperspirant composition prior to application to the skin of a consumer. When the particulate opacifying agent is included in the antiperspirant composition, minimized yellow color is achieved even when antiperspirant active compounds are included that may otherwise cause the antiperspirant compositions to exhibit a yellow color. More specifically, even when antiperspirant active compounds that reflect yellow light are present in the antiperspirant composition (and, thus, would normally cause the antiperspirant composition to have a yellow color), any such reflected yellow light is scattered to result in a white appearance of the antiperspirant compositions.

The antiperspirant active compound is based upon an astringent metallic salt. By “based upon”, it is meant that the antiperspirant active compound is either an astringent metallic salt, or is derived from an astringent metallic salt (such as a complex of an astringent metallic salt, buffer, and water). The antiperspirant active compound may be introduced into the antiperspirant composition through an antiperspirant active component that includes a separate buffer in addition to the antiperspirant active compound. Antiperspirant active compounds based upon astringent metallic salts are known in the art.

Examples of suitable antiperspirant active compounds include astringent salts of aluminum, zirconium, zinc, and mixtures thereof, which may be inorganic or organic salts. Some examples of inorganic and organic aluminum-containing and/or zirconium-containing salts include aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} x (H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. In an embodiment, the antiperspirant active compound includes zirconium salt complexes that additionally contain aluminum and glycine, commonly known as ZAG complexes. These ZAG complexes contain aluminum chlorohydroxide and zirconyl hydroxy chloride conforming to the above-described formulas. Examples of antiperspirant active compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof.

In an embodiment, the antiperspirant active compound is a salt of aluminum and zirconium having a ratio of aluminum to zirconium of greater than 6.0:1.0, such as from about 6.0:1.0 to 10.0:1.0. For example, in this embodiment, the antiperspirant active compound may include aluminum zirconium pentachlorohydrate, aluminum zirconium pentachlorohydrex glycine complex, or a combination of the aforementioned compounds. Aluminum zirconium pentachlorohydrex glycine complexes are commercially available, for example, from Summit Reheis of Huguenot, N.Y. under the designation AAZG-3110 as powder. In another example of this embodiment, the antiperspirant active compound may include aluminum zirconium octachlorohydrex glycine complex, commercially available, for example, from Summit Reheis under the designation AAZG-3109. The antiperspirant compositions described herein that include the salt of aluminum and zirconium having the ratio of aluminum to zirconium of greater than 6.0:1.0 may particularly benefit from the inclusion of the particulate opacifying agent because such salts may ordinarily cause the antiperspirant compositions to have a yellow color, and the particulate opacifying agent described herein can effectively minimize the yellow color and result in a whiter antiperspirant composition than could ordinarily be achieved when such salts are included.

The antiperspirant active compound is present in the antiperspirant composition in an amount that provides a measurable antiperspirant effect when applied to the underarms of a consumer. In an embodiment, the antiperspirant active compound is present in an amount of from about 10 to about 25 weight % (USP), such as from about 14 to about 20 weight % (USP), based on the total weight of the antiperspirant composition. As used herein, weight % (USP) of an antiperspirant compound is calculated as anhydrous weight percent in accordance with the U.S.P. method, as is known in the art. This calculation excludes any bound water and buffer such as glycine. It is to be appreciated that most commercially available antiperspirant active compounds are sold in mixtures along with other compounds that are not considered antiperspirant active compounds, such as water and buffer as described above in the context of the antiperspirant active component, and the above values do not account for the presence of such non-active compounds that may be present in the antiperspirant active component. In an embodiment, the antiperspirant active component includes antiperspirant active compounds in an amount of from about 30 to about 99 weight %, such as from about 50 to about 85 weight %, based on the total weight of the antiperspirant active component.

As set forth above, the antiperspirant composition also includes the particulate opacifying agent. Due to limits on the amount of antiperspirant compounds that can be included in antiperspirant compositions in accordance with government standards, and further due to the general desire to maximize antiperspirant efficacy of antiperspirant compositions, it is generally undesirable to reduce the amounts of the antiperspirant active compound within the antiperspirant compositions simply to improve aesthetic appearances, although such practices are common in the prior art. Reducing the amount of antiperspirant active compound may minimize the amount of white residue upon application; however, reducing the amount of antiperspirant active compound also reduces the amount of wetness protection delivered to consumers. The particulate opacifying agent is different from the antiperspirant active compound and has a refractive index that is greater than the refractive index of the antiperspirant composition in the absence of the particulate opacifying agent. As such, the particulate opacifying agent serves to raise the refractive index of the antiperspirant composition as a whole to thereby reduce a difference between refractive index of the antiperspirant active compound and the antiperspirant composition as a whole. By reducing the difference between refractive index of the antiperspirant active compound and the antiperspirant composition as a whole, appearance of white film is minimized upon application of the antiperspirant composition to skin of a consumer. At the same time, the difference in refractive index between the particulate opacifying agent and a comparable antiperspirant composition absent the particulate opacifying agent is less than or equal to about 0.3, such as less than or equal to about 0.2, to minimize appearance of white film upon application to skin of a consumer. As referred to herein, the “comparable antiperspirant composition absent the particulate opacifying agent” refers to an antiperspirant composition having the same formulation as the antiperspirant composition contemplated herein but without the particulate opacifying agent. To explain, the white film upon application to skin of a consumer is generally attributable to the disparity in refractive index between the antiperspirant composition and the antiperspirant active component. Should the particulate opacifying agent have a difference in refractive index from the antiperspirant composition in the absence of the particulate opacifying agent that is greater than about 0.3, the particulate opacifying agent itself could have a higher refractive index than the antiperspirant active compound and could aggravate appearance of white film upon application of the antiperspirant composition to skin of a consumer. Antiperspirant compositions generally have a refractive index of less than about 1.40 and, as set forth above, the antiperspirant active compounds generally have a refractive index of from about 1.45 to about 1.60. In an embodiment, the particulate opacifying agent has a refractive index of at least 1.40, such as from about 1.40 to about 1.60, at a wavelength of about 589 nm and a temperature of 21° C. Thus, while the particulate opacifying agent generally has a refractive index that is comparable to the antiperspirant active compound, the particulate opacifying agent can be employed to optimize aesthetics of the antiperspirant composition without adversely affecting antiperspirant efficacy of the antiperspirant composition.

As also alluded to above, due to the particulate form of the opacifying agent, the particulate opacifying agent scatters light to minimize yellow color of the antiperspirant composition prior to application to the skin of a consumer. To effectively scatter light, the particulate opacifying agent typically has an average particle size that is greater than the wavelengths of visible light, and typically has an average particle size that is substantially larger than the wavelengths of visible light. In an embodiment, the particulate opacifying agent has an average nominal particle size of from about 0.1 to about 0.5 μm, such as from about 0.1 to about 0.3 or from about 0.15 to about 0.20 μm.

The particular chemical composition of the particulate opacifying agent is not particularly limited so long as the refractive index provisions set forth above can be met. For example, the particulate opacifying agent may be chosen from the group of, but is not limited to, polymers such as styrene-containing copolymers, acrylate-containing copolymers, and combinations thereof; metal hydroxides; silicates; metal oxides; sulfates; amides; carbonates; phosphates; and combinations thereof. In an embodiment, the particulate opacifying agent includes a polymeric opacifying agent. In this regard, the particulate opacifying agent may be further distinguished from conventional metal oxide pigments due to the polymeric nature thereof. Examples of polymeric opacifying agents include, but are not limited to, styrene/acrylates copolymer, copolymers such as styrene/acrylates copolymer, acrylates/PEG-10 maleate/styrene copolymer, styrene/acrylamide copolymer, and combinations thereof. Such copolymers are commercially available under the tradename Opulyn™ from Dow Chemical Corporation of Midland, Mich. In a specific embodiment, the particulate opacifying agent includes a styrene/acrylates copolymer.

The particulate opacifying agent may be included in the antiperspirant compositions in any amount that results in a visible increase in whiteness of the antiperspirant composition, prior to application to skin of a consumer, as compared to an antiperspirant composition that is free of the particulate opacifying agent. In an embodiment, the particulate opacifying agent is present in the antiperspirant composition in an amount of from about 0.1 to about 8.0 weight %, such as from about 0.3 to about 6.0 weight %, based upon the total weight of the antiperspirant composition.

As set forth above, the antiperspirant composition may include additional components beyond the antiperspirant active compound and the particulate opacifying agent, such as the carrier system and the solidification system. The carrier system is not particularly limited, and suitable carriers that may be included in the carrier system are known in the art. For example, the carrier system may include one or more volatile silicone compounds. Examples of suitable silicone compounds that may be included in the carrier system include liquid siloxanes and, particularly, volatile polyorganosiloxanes having a measurable vapor pressure at ambient conditions. The polyorganosiloxanes can be linear or cyclic or combinations thereof. Exemplary siloxanes include cyclomethicones such as cyclotetrasiloxane, cyclopentasiloxane, cyclohexasiloxane, and combinations thereof. It is to be appreciated that the silicone carrier fluid may also comprise non-volatile silicones, which may be included for various skin-conditioning purposes. However, in an embodiment, such non-volatile silicones are present in a total amount of less than or equal to 1% by weight based upon the total weight of the antiperspirant composition to avoid excessive residual wetness after the antiperspirant composition dries. In an embodiment, the carrier system is present in the antiperspirant composition in an amount of from about 60 to about 85 weight %, for example, from about 65 to about 80 weight %, based upon the total weight of the antiperspirant composition.

The particular type of solidification system for the antiperspirant composition depends upon the form of the antiperspirant composition that is presented to the consumer. For example, the antiperspirant composition may be an anhydrous antiperspirant composition, and may include a wax component to provide a solid consistency to the antiperspirant composition. Such antiperspirant compositions are generally provided in solid stick form. The wax component includes any waxes that are present in the antiperspirant composition and may include one or more waxes that melt at a temperature of at least about 70° C., which are commonly referred to in the art as high melting point waxes. Suitable such waxes are well-known in the art. In addition to the wax component, other high melting components (i.e., components that melt at a temperature of at least about 70° C.) may be present in the antiperspirant composition including various types of high melting gelling agents such as polyethylene-vinyl acetate copolymers, polyethylene homopolymers, 12-hydroxystearic acid, and substituted and unsubstituted dibenzylidene alditols. In an embodiment, the high melting components (including the wax component) are present in the antiperspirant composition in an amount of from about 1 to about 25 weight %, such as from about 2 to about 15 weight %, based on the total weight of the antiperspirant composition.

In another embodiment, the antiperspirant composition is an oil and water emulsion, as may be the case for solid gel and extrudable gel forms of antiperspirant compositions. In these embodiments, the antiperspirant composition may be a water-in-oil emulsion or an oil-in-water emulsion, and may include a water phase and an oil phase. The water phase generally includes the antiperspirant active compound, water, and any other water-soluble compounds to be included in the antiperspirant composition. The oil phase generally includes the carrier system in addition to any other oil-soluble compounds (such as fragrance) that is to be included in the antiperspirant composition. In an embodiment, the water phase may be present in an amount of about 60 to about 85 weight % based upon the total weight of the emulsion, and the oil phase may be present in an amount of from about 15 to about 40 weight % based on the total weight of the emulsion. The antiperspirant composition in the form of the oil and water emulsion may have a soft, non-flowing, solid consistency (such as for solid gel forms) that can be rubbed or wiped across the skin, particularly the underarm. However, the various embodiments are not so limited and the antiperspirant composition can also have a soft gel, cream, or lotion consistency that is capable of extrusion.

Other components may also be present in the anhydrous antiperspirant compositions or oil and water emulsions as part of the carrier system, solidification system, or for other purposes known in the art. At least with regard to the anhydrous antiperspirant compositions, the other components mentioned below may be included in addition to or as an alternative to the wax component. Examples of other such components include, for example, non-volatile silicones, polyhydric alcohols having from 3-6 carbon atoms and from 2-6 hydroxyl groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, fumed silicas, and mixtures thereof. The term “fatty” is intended to include hydrocarbon chains of about 8 to 30 carbon atoms, preferably about 12 to 18 carbon atoms. In an exemplary embodiment, the antiperspirant composition comprises stearyl alcohol in an amount of from about 18 to about 22 wt. % of the antiperspirant composition.

For both the anhydrous and oil and water emulsion embodiments of the antiperspirant compositions, it is to be appreciated that additives other than the components mentioned above may also be included in the antiperspirant composition. The additives may include any of those used in conventional antiperspirant compositions. For example, the antiperspirant composition may include additives that cause the antiperspirant composition to exhibit long-lasting fragrance, odor protection, bacteria control, and/or another desired purpose and/or function. These additives include, but are not limited to, fragrances, including encapsulated fragrances, dyes, pigments, preservatives, antioxidants, skin conditioning agents such as moisturizers, and the like. The optional additives can be included in the antiperspirant composition in an amount of 0 to about 20 weight %.

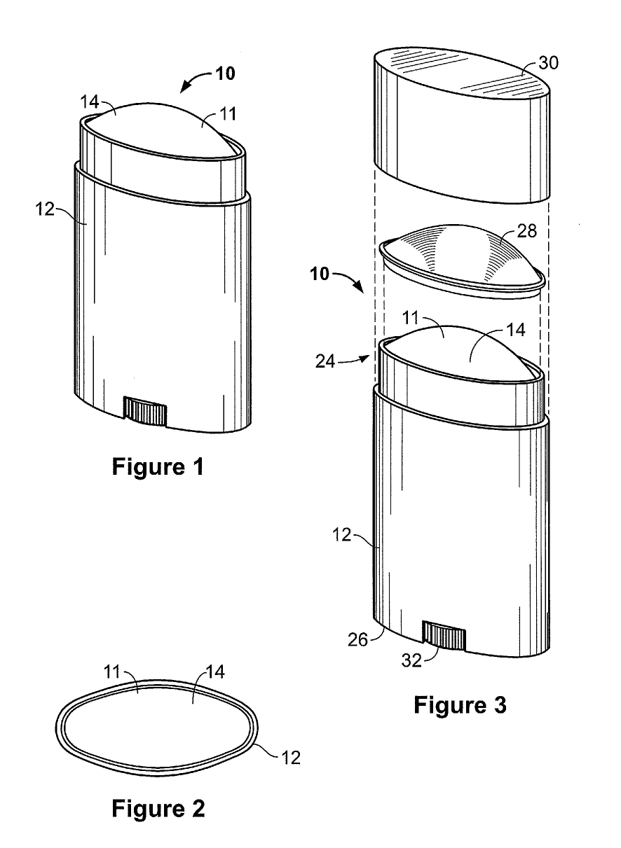

As set forth above, antiperspirant products are also provided herein. Referring to FIGS. 1-3, the antiperspirant products 10 include a container 12 and an antiperspirant composition 11 disposed in the container 12. Antiperspirant compositions that may be disposed in the container are described in detail above. In an exemplary embodiment, as best shown in FIG. 3, the container 12 has an application end 24 and an opposite end 26. A factory seal 28 may be positioned over an application surface 14 of the antiperspirant composition 11 to protect it during shipment and to render it tamper-proof prior to purchase. The factory seal 28 is removed by the user. The antiperspirant product 10 may also include a cover 30, and the cover 30 may be used to protect the antiperspirant composition 11 during storage of the antiperspirant product 10 between uses. Because the antiperspirant composition may be a solid stick, solid gel, or soft gel, the container generally includes an advancement device 32, e.g., a screw mechanism as shown, at the opposite end 26 of container 12 for moving the antiperspirant composition 11 upwards through the container 12 to continue to provide an exposed application surface. As the antiperspirant composition 11 is exhausted, it is advanced from the container 12 by the user using the advancement device 32. Such containers are well known in the art.

As set forth above, methods of preparing the antiperspirant compositions are also provided. The methods of preparing the antiperspirant compositions include the step of mixing the antiperspirant active compound and the particulate opacifying agent. The particular manner in which the antiperspirant compositions are prepared depends upon the type of solidification system used for the antiperspirant compositions. For example, when the solidification system includes the wax component, the antiperspirant active compound, wax component, and particulate opacifying agent, as well as any additional optional components, may be combined and mixed at elevated temperatures that are sufficient to melt the wax component and achieve a homogenous mixture of all components in the antiperspirant component. Alternatively, the antiperspirant active compound and wax may be combined and mixed at elevated temperatures and cooled to solidify the mixture. The mixture of the antiperspirant active compound and wax may be remelted, followed by mixing the particulate opacifying agent therein to form the antiperspirant composition.

In another embodiment, when the antiperspirant composition is an oil and water emulsion, a water phase including the antiperspirant active compound, particulate opacifying agent, and water (as well as additional optional water-soluble components) can be prepared by combining and heating the components of the water phase under mixing. A separate oil phase including water-insoluble components, such as volatile silicones, can be prepared by combining and heating the components of the oil phase under mixing. The water phase and oil phase can then be mixed to form the antiperspirant composition in oil and water emulsion form. Additional components, such as fragrance, can be mixed into the oil and water emulsion.

The following Examples are to be read as illustrative of the antiperspirant compositions contemplated herein and method of making the same as described herein, and are not to be interpreted as limiting.

### EXAMPLES

### Preparation I

Antiperspirant Composition A, an anhydrous antiperspirant composition in solid stick form, was prepared by providing a Base Antiperspirant Composition that includes the components set forth in Table I, absent the particulate opacifying agent, melting the Base Antiperspirant Composition, and mixing the particulate opacifying agent into the Base Antiperspirant Composition to form Antiperspirant Composition A. The Antiperspirant Composition A was then cooled, and whiteness of Antiperspirant Composition A was observed as compared to the Base Antiperspirant Composition. Antiperspirant Composition A was visibly whiter than the Base Antiperspirant Composition. Table I provides the types and amounts of each component included within the Base Antiperspirant Composition and Antiperspirant Composition A, with all amounts in weight % based upon the total weight of the respective antiperspirant compositions.

TABLE I

| | Base Antipers. | Antipers. |
|---|---|---|
| | Comp. | Comp. A |
| Cyclopentasiloxane | 38.49 | 36.57 |
| Antiperspirant Active | 23.68 | 22.49 |
| Component A | | |
| Thickening Agent A | 0.32 | 0.30 |
| Thickening Agent B | 1.35 | 1.28 |
| Wax | 2.84 | 2.70 |
| Stearyl Alcohol | 20.00 | 19.00 |
| PAG-based Fluid | 9.80 | 9.31 |
| Particulate Opacifying Agent | 0.00 | 5.00 |
| Additive A | 1.92 | 1.82 |
| Additive B | 1.50 | 1.43 |
| Additive C | 0.10 | 0.10 |
| Total | 100.00 | 100.00 |

Antiperspirant Active Component A is a complex of an aluminum and zirconium halide salt and glycine having a ratio of aluminum to zirconium of from about 6.0:1.0 to about 10.0:1.0, provided in powder form.

Thickening Agent A is a hydrophilic fumed silica with a specific surface area of 300 m2/g.

Thickening Agent B is a hydrophobic fumed silica with a specific surface area of 130 m2/g.

Wax is a hydrogenated castor wax.

PAG-based Fluid is a modified polyalkylene glycol.

Particulate Opacifying Agent is an anionic styrene/acrylic copolymer supplied at 40% solids with a nominal average particle size of about 0.18 μm, commercially available as Opulyn™ 301 from Dow Chemical Corporation.

Additive A is myristyl myristate skin conditioner.

Additive B is perfume.

Additive C is perfume.

The higher refractive index of Antiperspirant Composition A as compared to the Base Antiperspirant Composition shown in Table I resulted in a more closely matched refractive index of the Antiperspirant Composition A to the refractive index of the antiperspirant active component therein and, thus, reduced the appearance of white film upon application to skin as compared to the Base Antiperspirant Composition.

### Preparation II

Antiperspirant Composition B was prepared as a water-in-oil emulsion. In particular, all components of a water phase were combined and heated under mixing. All components of an oil phase were separately mixed from the water phase and heated under mixing. The water phase was then mixed with the oil phase under continued mixing until a microscopically homogeneous emulsion was achieved. Fragrance was then added to form the Antiperspirant Composition B, which was then mixed for about 5 minutes at from 12,000 to 15,000 rpm. Table II provides the types and amounts of each component included within Antiperspirant Composition B, with all amounts in weight % based upon the total weight of the Antiperspirant Composition B.

TABLE II

| | | Antipers. Comp. B |
|---|---|---|
| Water Phase | Water | 10.87 |
| | Antiperspirant | 54.91 |
| | Active Component B | |
| | Propylene Glycol | 5.63 |
| | Particulate | 0.50 |
| | Opacifying Agent | |
| | Co-Solvent | 10.94 |
| Oil Phase | Silicone A | 9.05 |
| | Silicone B | 7.45 |
| Additive D | | 0.65 |
| | | 100.00 |
| Refractive Index, No | | ??? |
| Particulate Opacifying | | |
| Agent | | |

Antiperspirant Active Component B is a mixture of an aluminum and zirconium halide salt having a ratio of aluminum to zirconium of from about 6.0:1.0 to about 10.0:1.0, glycine, and water, with a % activity of about 29.2%.

Co-Solvent is a modified ethyl alcohol.

Silicone A is a cyclomethicone and dimethicone copolyol.

Silicone B is a dimethicone.

Additive D is a fragrance.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. An antiperspirant composition comprising:
an antiperspirant active compound based upon an astringent metallic salt; and
a particulate opacifying agent having a refractive index greater than a comparable antiperspirant composition absent said particulate opacifying agent, provided that a difference in refractive index between said particulate opacifying agent and said comparable antiperspirant composition absent said particulate opacifying agent is less than or equal to about 0.3.

2. The antiperspirant composition of claim 1, wherein said antiperspirant active compound is chosen from salts of aluminum, zirconium, zinc, and mixtures thereof.

3. The antiperspirant composition of claim 2 wherein said antiperspirant active compound is a salt of aluminum and zirconium having a ratio of aluminum to zirconium of greater than 6.0:1.0.

4. The antiperspirant composition of claim 1, wherein said particulate opacifying agent comprises a polymeric opacifying agent.

5. The antiperspirant composition of claim 4, wherein said particulate opacifying agent comprises a styrene/acrylates copolymer.

6. The antiperspirant composition of claim 1, wherein said particulate opacifying agent has a refractive index of at least 1.4 at a wavelength of about 589 nm at a temperature of 21° C.

7. The antiperspirant composition of claim 1, wherein said particulate opacifying agent has an average nominal particle size of from about 0.1 to about 0.5 μm.

8. The antiperspirant composition of claim 1, wherein said particulate opacifying agent is present in an amount of from about 0.1 to about 8.0 weight %, based upon the total weight of said antiperspirant composition.

9. The antiperspirant composition of claim 1, further defined as an anhydrous antiperspirant composition.

10. The antiperspirant composition of claim 9, further comprising a wax component.

11. The antiperspirant composition of claim 1, further defined as an oil and water emulsion.

12. An antiperspirant product comprising:
a container; and
an antiperspirant composition disposed in said container and comprising:
an antiperspirant active compound based upon an astringent metallic salt; and
a particulate opacifying agent having a refractive index greater than a comparable antiperspirant composition absent said particulate opacifying agent, provided that a difference in refractive index between said particulate opacifying agent and said comparable antiperspirant composition absent said particulate opacifying agent is less than or equal to about 0.3.

13. An antiperspirant product as set forth in claim 12, wherein said antiperspirant active compound is chosen from salts of aluminum, zirconium, zinc, and mixtures thereof

14. The antiperspirant product of claim 13 wherein said antiperspirant active compound is a salt of aluminum and zirconium having a ratio of aluminum to zirconium of greater than 6.0:1.0.

15. The antiperspirant product of claim 12, wherein said particulate opacifying agent has a refractive index of at least 1.4 at a wavelength of about 589 nm at a temperature of 21° C.

16. The antiperspirant product of claim 12, wherein said particulate opacifying agent has an average nominal particle size of from about 0.1 to about 0.5 μm.

17. A method of preparing an antiperspirant composition, said method comprising the step of mixing:
an antiperspirant active compound based upon an astringent metallic salt; and
a particulate opacifying agent having a refractive index greater than a comparable antiperspirant composition absent said particulate opacifying agent, provided that a difference in refractive index between said particulate opacifying agent and said comparable antiperspirant composition absent said particulate opacifying agent is less than or equal to about 0.3 compound and a first buffer.

18. The method of claim 17, wherein the antiperspirant active compound is a salt of aluminum and zirconium having a ratio of aluminum to zirconium of greater than 6 01 0

19. The method of claim 17, wherein the particulate opacifying agent has a refractive index of at least 1.4 at a wavelength of about 589 nm at a temperature of 21° C.

20. The method of claim 17, wherein the particulate opacifying agent has an average nominal particle size of from about 0.1 to about 0.5 μm.
